(19) United States
(12) Patent Application Publication
(10) Pub. No.: US 2024/0277746 A1
(43) Pub. Date: Aug. 22, 2024

(54) OPHTHALMIC NANO-DISPERSION COMPOSITION, A METHOD OF PREPARATION THEREOF, AND FORMULATION FOR CORNEAL EPITHELIAL WOUND HEALING

(71) Applicant: THE UNIVERSITY OF JORDAN, Amman (JO)

(72) Inventors: Hatim ALKHATIB, Amman (JO); Mai JABER, Amman (JO); Bahaa Al Din JABER, Amman (JO)

(73) Assignee: THE UNIVERSITY OF JORDAN, Amman (JO)

(21) Appl. No.: 18/571,296

(22) PCT Filed: Jun. 17, 2021
(86) PCT No.: PCT/JO2021/050007

§ 371 Date: Dec. 18, 2023

(51) Int. Cl. A61K 31/7048; A61K 9/00; A61K 9/10; A61K 31/375; A61K 47/44; A61P 27/02

(52) U.S. Cl. CPC A61K 31/7048; A61K 9/0048; A61K 9/10; A61K 31/375; A61K 47/44; A61P 27/02

(57) ABSTRACT

Provided is a pharmaceutical solid-in-oil nano-dispersion composition that may include ascorbic acid or a astable derivative thereof, a surfactant coating the ascorbic acid or its derivative and an oil vehicle, wherein the ascorbic acid or its derivative are uniformly dispersed in the solid-in-oil nano-dispersion composition. In addition to that, the present disclosure provides a method of preparing the solid-in-oil nano-dispersion composition and formulations thereof. Also, after administration the S/O nano-dispersion based dosage form to subject, it will improve the corneal epithelial wounds healing.

### TECHNICAL FIELD

The present disclosure relates to ophthalmic compositions and formulations, and more particularly to solid-in-oil nano-dispersion ophthalmic compositions and formulations that improve the chemical stability and the bioavailability of ascorbic acid for faster corneal epithelial wound healing.

### BACKGROUND

Corneal wound healing is a complex process that involves cellular changes, signaling molecules from cells of every layer of the cornea, changes in the composition and configuration of the extracellular matrix (“ECM”) and corneal neovascularization.

High concentrations of ascorbic acid. (“AA”) (Vitamin C) are found in various ocular fluids and tissues, including the cornea. In humans, the concentration of AA in corneal epithelium is 300 times higher than that in the plasma. This suggests a significant role of AA in the function of corneal epithelium.

AA has been shown to play a role in synthesizing parallel arrays of ECM fibrils in cultured human keratocytes. It is also known to be involved in the suppression of corneal neovascularization via its antioxidant activity and ability to enhance collagen synthesis. It has also been proven to accelerate corneal epithelial wound healing, promote the stemness of corneal epithelial stem/progenitor cells, and promote ECM production.

AA, a water-soluble vitamin, has a variety of biological and pharmaceutical functions. These functions are closely related to the well-known antioxidant properties of this compound. AA, however, is very unstable to oxygen, moisture, light, heat, metal ions, and basic environments easily decomposing into biologically inactive compounds such as 2,3-diketo-Lgulonic acid, oxalic acid, L-threonic acid, L-xylonic acid, and L-Lyxonic acid.

Solid-in-oil nano-dispersions (“S/O”) are unique formulations that can improve the dispersibility of hydrophilic solutes in an oily phase. This system presents the drug in the solid form that is nano-coated with lipophilic surfactant molecules and dispersed in oily phase. S/O nano-dispersions provide small, hydrophobically-modified nanoparticles that can improve the permeation of hydrophilic drugs through hydrophobic biological barriers.

For instance, the international patent application published under number WO2017030328 discloses a corneal damage prevention/treatment composition including thymosin beta 4 and citric acid or salts of same. The composition can additionally include any organic acid selected from acetic acid, ascorbic acid or salts of same. The composition enables the activity of existing thymosin beta 4 to be maintained or increased and thus provides effective treatment for corneal wound. Therefore, the composition can be used as an ophthalmic formulation for corneal damage treatment.

The U.S patent application published under number US20140141066 discloses an encapsulated ascorbic acid composition that protects the encapsulated ascorbic acid from unwanted interactions and significantly reduces the likelihood of an unwanted allergic reaction for many users. The encapsulated ascorbic acid composition accomplishes this using sunflower lecithin. Sunflower lecithin is utilized as a carrier material that forms a liposomal barrier surrounding ascorbic acid. Sunflower lecithin does not contain common allergens, allowing for increased accessibility of the encapsulated ascorbic acid composition. Additionally, the sunflower lecithin contains a higher quantity of the phospholipid, phosphatidylcholine, compared to other lecithin sources. Moreover, phosphatidylcholine increases the bioavailability of ascorbic acid.

The prior art ophthalmic drug delivery systems may carry the potential of a low local concentration of the drug in its site of action caused by the complicated anatomical structure of the eye, the small absorptive surface and low permeability of the cornea, the continuous tear formation, and the constant blinking and flow of substances through nasolacrimal duct. The present disclosure provides pharmaceutical composition, method of preparation thereof, and a formulation that achieves the full potential of ophthalmic drug delivery system, through the S/O formulations, which can improve corneal bioavailability of topically applied hydrophilic drug. In addition, it can improve the chemical stability of AA.

### SUMMARY

It is an object of the present disclosure to develop and evaluate an effective ophthalmic S/O nano-dispersion composition of AA or a stable derivative thereof to accelerate corneal epithelial wound healing.

It is an object of the present disclosure to improve the bioavailability of AA after topical ophthalmic application for faster corneal epithelial regeneration.

It is another object of the present disclosure to utilize S/O nano-dispersion to prepare an ophthalmic dosage form.

In aspects of the present disclosure, the S/O nano-dispersion composition may include AA or a chemically stable derivative thereof; a hydrophobic surfactant; and an oil vehicle.

In some aspects, the hydrophobic surfactant may be Span 85.

In some aspects, the oil vehicle may be castor oil.

In some aspects of the present disclosure, the stable derivative of AA may be Ascorbyl glucoside (“AA-2G”).

Further aspects of the present disclosure provide a method of preparing AA and AA-2G S/O nano-dispersion composition.

Aspects of the present disclosure further provide a formulation of a composition of the S/O nano-dispersion as an ophthalmic dosage form.

In some aspects, ophthalmic dosage form can be formulated as an oily eye drops or a solid-in-oil in water (“S/O/W”) emulsion.

Further aspects of the present disclosure provide a method of preparing oily eye drops and a solid in oil in water S/O/W emulsion.

In aspects of the present disclosure, S/O nano-dispersion can enhance the transcorneal permeability of AA and AA-2G.

In another aspect, S/O nano-dispersion can improve the accumulation of AA and AA-2G within the cornea.

In aspects of the present disclosure, the AA and AA-2G loaded S/O nano-dispersion based dosage form can accelerate corneal epithelial wound healing.

The details of one or more embodiments of the disclosure are set forth in the drawings and description below. Other features, objects, and advantages of the disclosure will be apparent from the description and from the claims. All references cited herein are hereby incorporated by reference in their entirety.

### BRIEF DESCRIPTION OF THE DRAWINGS

The present disclosure will now be described with reference to the accompanying drawings, without however limiting the scope of the disclosure thereto, and in which:

FIG. 1 illustrates a flowchart of a method for preparing a pharmaceutical S/O nano-dispersion composition configured in accordance with embodiments of the present disclosure.

FIG. 2A illustrates transmission electron microscopy (“TEM”) image of the pharmaceutical S/O nano-dispersion composition formulated using about 0.5 g of Span 85, the composition being prepared in accordance with embodiments of the present disclosure.

FIG. 2B illustrates TEM image of the pharmaceutical S/O nano-dispersion composition formulated using about 1.0 g of Span 85, the composition being prepared in accordance with embodiments of the present disclosure.

FIG. 2C illustrates TEM image of the pharmaceutical S/O nano-dispersion composition formulated using about 1.2 g of Span 85, the composition being prepared in accordance with embodiments of the present disclosure.

FIG. 2D illustrates TEM image of the pharmaceutical S/O nano-dispersion composition formulated using about 1.5 g of Span 85, the composition being prepared in accordance with embodiments of the present disclosure.

FIG. 3 illustrates the effects of the weight ratio between Span 85 and AA-2G on the corneal permeation of AA-2G (Q6 hr) in a pharmaceutical S/O nano-dispersion composition prepared in accordance with embodiments of the present disclosure.

FIG. 4 illustrates the effects of the weight ratio between Span 85 and AA-2G on the corneal accumulation of AA-2G (Qr6 hr) pharmaceutical S/O nano-dispersion composition prepared in accordance with embodiments of the present disclosure.

FIG. 5 illustrates a flowchart of a method for preparing S/O/W emulsion comprising the pharmaceutical S/O nano-dispersion composition configured in accordance with embodiments of the present disclosure.

FIG. 6 illustrates a representative post-surgical fluorescein staining taken at indicated time points of rabbit corneas subjected to epithelial debridement and allowed to heal after treatment of normal saline (Control), 5% AA solution or AA loaded S/O nano-dispersion composition prepared in accordance with embodiments of the present disclosure over a 72-hour period.

FIG. 7 illustrates corneal epithelial wound healing percent over time on denuded corneas treated with normal saline or 5% ascorbic acid solution. (mean±SD, n=4).

FIG. 8 illustrates corneal epithelial wound healing percent over time on denuded corneas treated with normal saline or AA loaded S/O nano-dispersion. (mean±SD, n=4).

FIG. 9 illustrates corneal epithelial wound healing percent over time on denuded corneas treated with normal saline or AA-2G loaded S/O nano-dispersion. (mean±SD, n=4).

FIG. 10 illustrates normalized healing rates of AA solution or S/O nano-dispersion to normal saline healing rate over a 72-hour period post corneal debridement, p value after 6 hours and p value after 12 hours. (mean±SD, n=4). The table represents the significance levels based on the ANOVA analysis.

### DETAILED DESCRIPTION

Embodiments of the present disclosure provide a pharmaceutical S/O ophthalmic nano-dispersion composition and formulations of the composition, namely oily drops, ointments, and S/O/W emulsion.

In embodiments of the present disclosure, the S/O ophthalmic nano-dispersion composition may include AA or a stable derivative thereof, a hydrophobic surfactant coating the AA or its derivative and an oil vehicle, wherein the AA or its derivative are uniformly dispersed in the oily vehicle.

In some embodiments of the present disclosure, the stable derivative of AA may be AA-2G.

In embodiments of the present disclosure, hydrophobic surfactants may be selected from a group including spans, sucrose fatty acid esters, or combinations thereof.

In some embodiments, the hydrophobic surfactant may be Span 85.

In embodiments of the present disclosure, the oil vehicle may be selected from soya oil, castor oil, medium chain triglycerides (“MCT”), long chain triglycerides (“LCT”), Myglyol 812 (triesters of glycerol, capric and caprylic acids), mineral oil, isopropyl myristate, or combinations thereof.

Embodiments of the present disclosure further provide a method of preparing the ophthalmic S/O nano-dispersion composition using difference levels of Span 85 (0.5, 1, 1.2, or 1.5 g).

The nanoparticles prepared had a diameter of less than 40 nm having an excellent encapsulation effectively (EE>99%). They were also stable in term of particle size, polydispersity index and encapsulation efficiency.

The S/O nano-dispersion resulted in improved permeation (up to 18×) and accumulation (up to 7×) of AA or AA-2G in transcorneal diffusion experiments in comparison to AA or AA-2G solutions, receptively.

Embodiments of the present disclosure further provide formulations of the S/O ophthalmic nano-dispersion composition in the form of oily drops, S/W/O emulsions, or ointments.

In embodiments of the present disclosure, the S/O/W ophthalmic composition may comprise S/O nano-dispersions as an oily phase stabilized by a non-ionic hydrophilic surfactant to form an oil in water (O/W) emulsion.

In embodiments of the present disclosure, the hydrophilic surfactants may be selected from a group including Tween 80, Poloxamer 188, lecithin, or combinations thereof. An auxiliary emulsifier may be used to stabilize the emulsion and change the surface properties of the emulsions in term of zeta potential such as chitosan, methyl cellulose, carboxymethyl cellulose, carrageenan, or combinations thereof.

In embodiments of the present disclosure, S/O nano-dispersion loaded with AA or AA-2G provides small nanoparticles hydrophobically modified that can penetrate through the cells given improvement in corneal epithelial healing in-vivo in comparison to AA solutions.

### Example 1

### Preparation of S/O Nano-Dispersion

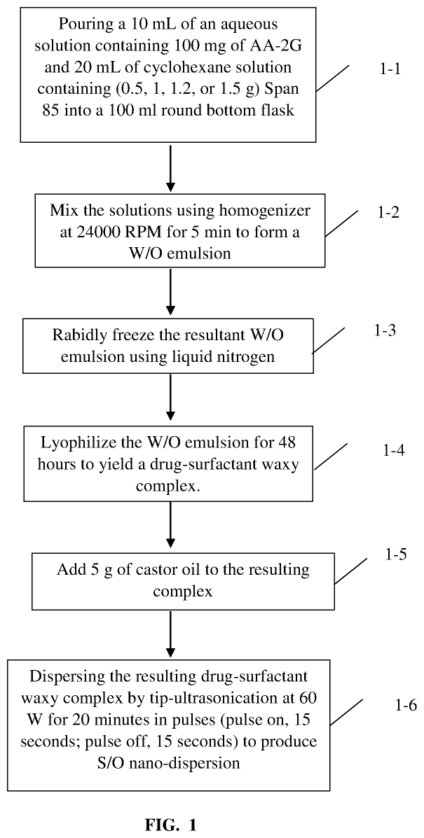

Reference in this example is being made to FIG. 1, which illustrates a method of preparing S/O nano-dispersion in accordance with embodiments of the present disclosure. According to the method, about 10 mL of an aqueous solution containing about 100 mg AA-2G or about 52 mg AA and about 20 mL cyclohexane solution containing (0.5, 1, 1.2, or 1.5 g) Span 85 were poured into a 100 mL round-bottom flask (process block 1-1) and mixed using a homogenizer at about 24000 rpm for about 5 min to form a water-in-oil (W/O) emulsion (process block 1-2). The resulting emulsion was rapidly frozen in liquid nitrogen (process block 1-3) and lyophilized for about 48 hours to yield a drug-surfactant waxy complex (process block 1-4). Then, about 5 g of castor oil was added to the resulting complex (process block 1-5) and thoroughly dispersed by probe-ultrasonication at about 60 W for about 20 minutes in pulses (pulse on, 15 seconds; pulse off, 15 seconds) to produce S/O nano-dispersion (process block 1-6).

During the ultrasonication process, an ice bath was used to avoid temperature elevation.

In the method of the present disclosure, theoretical AA and AA-2G concentration was set to about 8 mg/ml, 16 mg/ml, respectively.

Particle size distribution and polydispersity index for each nano-dispersion were measured using dynamic light scattering (“DLS”) in a Zetasizer Nano ZS (Malvern, Worcestershire, UK.) wherein regardless of the concentration of Span 85, all S/O nano-dispersion had a mean particle size in the range of from about 33.5 nm to about 43.8 nm with polydispersity indices between about 0.237 and about 0.314, which makes it suitable for the ocular application.

The small size lends the particles easily to cellular uptake providing a potential for overcoming biological and physical barriers that currently limit the clinical translation of drug. No significant differences were detected between the four formulas (p value>0.05).

The process was effective in transforming AA-2G or AA into nano-dispersed particles which is reflected by the encapsulation efficiency (“EE”) obtained for the different formulations which was higher than 99.22%. No significant difference was detected between the four formulas (p value>0.05).

Stability of S/O nano-dispersion during storage at 4° C. for 60 days was evaluated by visual observation of crystal precipitation, particle size and PDI measurements using dynamic light scattering as well as EE % as a measure of the leakage of AA or AA-2G into the oil phase.

No formulation showed crystal formation or precipitation throughout a 60 days' duration, no significant change was observed in particle size and polydispersity index (“PDI”) of S/O nano-dispersion. Also, no significant variation was observed in EE %.

All S/O formulations are considered to be stable without a tendency to either aggregation or particle disruption.

### Example 2

### Morphological Microscopic Examination (Transmission Electron Microscopy)

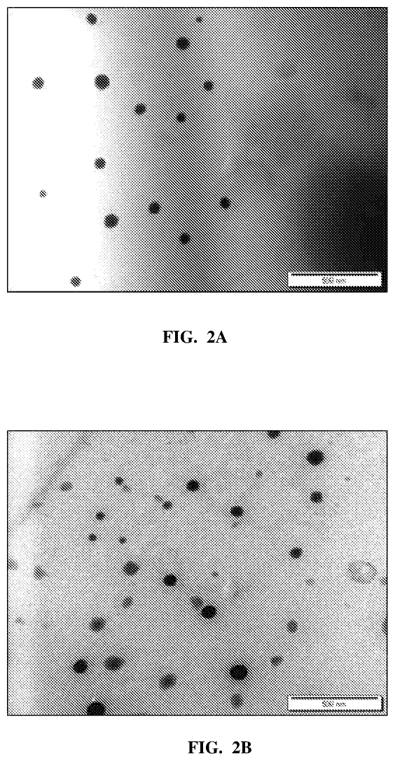

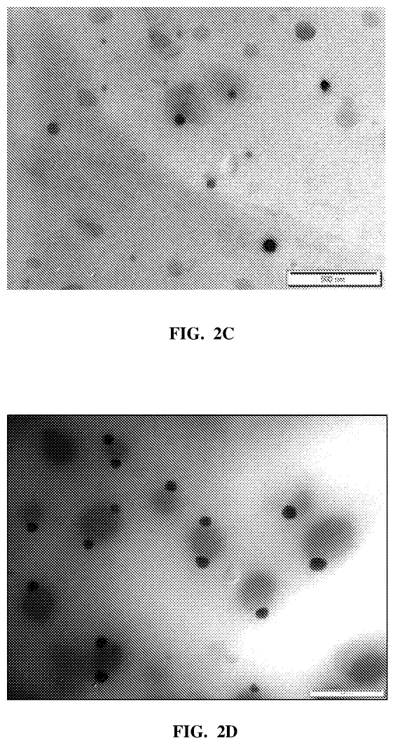

TEM was used to determine the size and shape of the synthesized nanoparticles. Nano-sized particle formation in the S/O dispersions was confirmed by size distribution analysis using morphological investigation (TEM). The formation of spherical objects was clearly seen in TEM images (FIGS. 2A-2D) with narrow size distribution. Results show that there is no significant difference in particle size and shape between different formulas.

### Example 3

### Ex Vivo Study

### Permeation/Accumulation Study

In vitro permeation and accumulation studies were performed on freshly excised sheep cornea obtained from a local slaughterhouse within an hour after the slaughter of the animals.

After the preparation of excised cornea, it was directly fixed between the donor and receptor compartments of Franz-type diffusion cells in such a way that its epithelial surface was facing the donor compartment and the endothelial surface was facing the receptor compartment.

The diameter of the diffusion area of the diffusion cells was 11 mm, producing an effective diffusion area of about 0.95 cm². The receptor compartment was filled with about 12 ml of normal saline solution stirred using teflon-coated magnetic stirring bars and maintained at a temperature of about 37° C. by circulating warm water through the water jacket around the receptor compartment. Special care was taken to expel air bubbles from the receptor fluid.

The permeation study was carried out by placing about 1 ml of either the prepared AA-2G loaded S/O nano-dispersion, AA-2G solution or normal saline on top of the cornea in the donor compartment.

An aliquot (about 0.3 ml) of the receptor fluid was withdrawn at about 0.5, 1, 1.5, 2, 3, 4, and 6 hours after beginning the experiment and replaced by an equal volume of fresh receptor solution. The receptor fluid content of AA-2G was determined quantitatively using the High-performance liquid chromatography (“HPLC”) method.

To quantify the amount of AA-2G entrapped within the corneal tissue after finishing the in vitro permeation study, the diffusion cells were dismantled, the corneas were quickly washed with normal saline to remove the surface-attached AA-2G, then the diffusional areas were cut out and digested using about 0.5 ml of 0.5 M NaOH for about 12 hours at about 37° C. in a shaker incubator to obtain a solution. After that, about 0.5 ml of ethanol and about 1 ml of acetonitrile were added to this solution to precipitate the dissolved proteins. The samples were then centrifuged at about 8000 rpm for about 10 minutes. The supernatants were taken and diluted with the mobile phase for HPLC analysis.

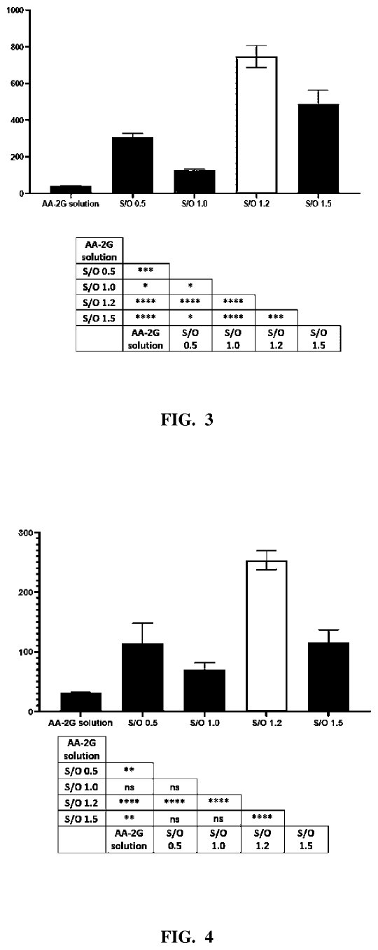

The results of transcorneal permeation studies with different S/O nano-dispersion formulations have clearly demonstrated their effectiveness in accelerating the permeation of AA-2G through excised sheep corneas. The results are summarized in Table (1) and FIG. 3.

TABLE 1
Amount permeated/accumulated per unit area Qt, Qr in μg/cm2, (mean ± SD, n = 3)

| | AA-2G | | | | |
|---|---|---|---|---|---|
| Time (hr) | solution | S/O 0.5 | S/O 1.0 | S/O 1.2 | S/O 1.5 |
| 0.5 | 0.00 | 0.00 | 15.8 ± 14.82 | 8.78 ± 13.26 | 5.17 ± 5.97 |
| 1 | 0.00 | 4.88 ± 1.73 | 23.47 ± 16.01 | 23.63 ± 16.84 | 10.76 ± 12.56 |
| 1.5 | 0.00 | 24.83 ± 5.04 | 31.45 ± 19.71 | 60.13 ± 31.57 | 21.63 ± 24.13 |
| 2 | 0.00 | 47.48 ± 9.77 | 45.57 ± 17.17 | 108.38 ± 48.24 | 45.04 ± 38.23 |
| 3 | 4.56 ± 3.29 | 78.03 ± 37.15 | 61.57 ± 8.21 | 232.11 ± 64.77 | 124.38 ± 49.11 |
| 4 | 11.21 ± 3.67 | 201.95 ± 19.52 | 81.83 ± 4.25 | 413.59 ± 103.19 | 228.57 ± 82.25 |
| 6 | 41.02 ± 0.21 | 307.98 ± 19.64 | 123.83 ± 7.29 | 747.69 ± 60.07 | 486.58 ± 75.62 |
| Enhancement | | 7.51 | 3.02 | 18.23 | 11.86 |
| Ratio | | | | | |
| permeation | | | | | |
| accumulation | 30.91 ± 1.04 | 130.78 ± 53.99 | 70.43 ± 11.05 | 218.48 ± 48.96 | 116.34 ± 20.60 |
| Enhancement | | 4.23 | 2.28 | 7.07 | 3.76 |
| Ratio | | | | | |
| accumulation | | | | | |

According to FIG. 3 and Table (1), the permeation of AA-2G (Qt-6 hr) was in the following order: S/O 1.2>S/O 1.5>S/O 0.5>S/O 1.0>AA-2G solution. S/O nano-dispersion significantly enhanced transcorneal permeability of AA-2G in comparison with simple aqueous solution of AA-2G with a p value of 0.0009, 0.0451, <0.0001 and <0.0001 for S/O 0.5, 1.0, 1.2, 1.5 nano-dispersion respectively. The permeation-enhancement ratios were 7.51, 3.02, 18.23, 11.86 for S/O 0.5, 1.0, 1.2, 1.5 nano-dispersion respectively.

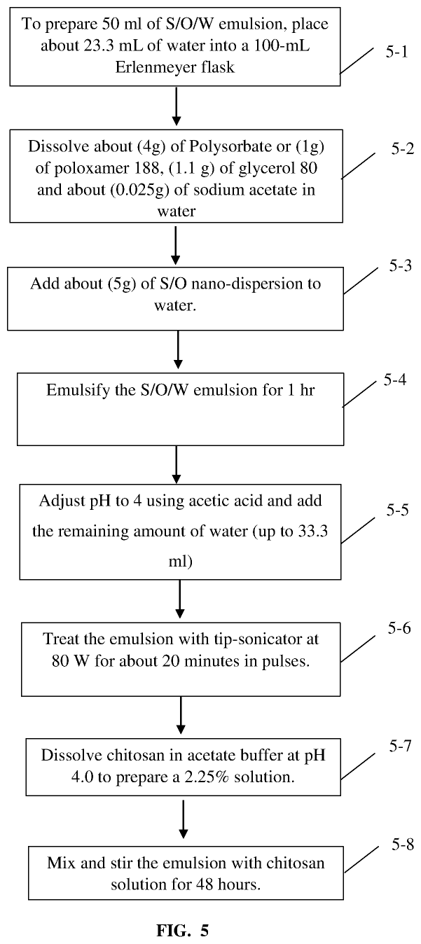

The results of FIG. 4 and Table (1) of accumulation studies with different formulations indicate that the S/O nano-dispersion effectively increased the accumulation of AA-2G in the corneal tissue. The accumulation of AA-2G (Qr6 hr) was in the following order: S/O 1.2>S/O 1.5˜S/O 1.0˜S/O 0.5>AA-2G solution. S/O nano-dispersion significantly improved corneal accumulation of AA-2G in comparison to simple aqueous solution of AA-2G with p value of 0.0041, 0.1208, <0.0001 and 0.0015 for S/O 0.5, 1.0, 1.2, 1.5 nano-dispersion in comparison to the AA-2G solution respectively. The accumulation-enhancement ratios were 4.23, 2.28, 7.07, 3.76 for S/O 0.5, 1.0, 1.2, 1.5 nano-dispersion respectively.

Marked increase in the permeation and accumulation of AA-2G were found upon transcorneal application of S/O 1.2. These results could be rationalized considering that a higher Span 85 to AA-2G ratio produced hydrophobic nanoparticles with better surfactant surface coverage. Which can effectively promote AA-2G delivery into and through the hydrophobic corneal barrier. However, further increase in the weight ratio (S/O 1.5) reduced both penetration of AA-2G into and across the cornea. Increasing in the Span 85 concentration in the formulation to the excess level encourages the partitioning of AA-2G nanoparticles into the castor oil rather than the partitioning into the corneal layers.

Based on the results, it was concluded that the optimal weight ratio for the highest AA-2G corneal permeation and accumulation was 1.2 of Span 85 to 0.1 of AA-2G. The mean diameter of the S/O nano-dispersion was about 43.4 nm and the encapsulation efficiency was 99.37% with good stability over 2 months.

### Example 4

### Utilizing S/O Nano-Dispersion to Prepare Eye Drops

Based on the permeation and penetration parameters, the most effective S/O nano-dispersion was selected to prepare the final eye drop dosage as either an oily eye drop, or as S/O/W emulsions.

Oily eye drop was prepared by simple mixing about 5 g S/O nano-dispersion and about 5 g MCT (Crodamol® GTC/C). In blank oily eye drop, about 5 g castor oil was used instead of S/O nano-dispersion.

Particle size and PDI were measured before and after mixing of S/O nano-dispersion with MCT. The results indicated that particle size was about 40 nm with no significant difference from castor oil-based S/O nano-dispersion.

S/O/W emulsions were prepared using two different types of nonionic surfactants, tween 80 or poloxamer 188.

Reference now is being made to FIG. 5, which illustrates a method of preparing oil-in-water emulsion in accordance with embodiments of the present disclosure. The method may include the steps of:
Putting about 75% of the required amount of water into a flask (process block 5-1);
Dissolving the required amount of hydrophilic surfactant, glycerol and sodium acetate in the water (process block 5-2);
Adding the previously prepared S/O nano-dispersion to the water (process block 5-3);
Emulsifying the S/O nano-dispersion using a homogenizer (IKA, T18 B, Germany) at about 24000 rpm for about 1 hour (Process block 5-4);
Adjusting pH to about 4.0 using acetic acid and adding the remaining amount of water (process block 5-5);
Treating the emulsion with tip sonication at 80 W for about 20 minutes in pulses (pulse on, 60 seconds; pulse off, 30 seconds) (process block 5-6);
Dissolving Low Molecular Weight Chitosan in acetate buffer at pH 4.0 (process block 5-7); and
Mixing and stirring the emulsion with chitosan solution for about 48 hours (process block 5-8).

The hydrodynamic mean particle sizes of emulsions were measured using dynamic light scattering. Also, surface charge properties of the emulsion droplets were evaluated by measuring the zeta potential.

The results of Table (2) show the mean droplet diameter and zeta potential of the O/W nanosized emulsions. The particle size distribution of the emulsion can reflect the droplet flocculation and aggregation. The mean droplet diameter for Tween 80 based emulsions was in the range of from about 150 nm to about 170 nm while it was about 400 nm for Poloxamer 188 based emulsions.

For the polydispersity indices, all emulsions had values lower than 0.3, which indicated that all emulsions formed uniform droplets and presented a narrow droplet size distribution.

TABLE 2
Particle size diameter and zeta potential of emulsions

| | | | Zeta potential |
|---|---|---|---|
| | size (nm) | PDI | (mV) |
| Blank O/W-T | 168.1 ± 2.664 | 0.244 ± 0.044 | 22.8 ± 0.696 |
| S/O/W-T | 156.9 ± 1.55 | 0.289 ± 0.002 | 20.8 ± 0.3 |
| Blank O/W-P | 394.9 ± 51.03 | 0.297 ± 0.066 | 15.3 ± 0.61 |
| S/O/W-P | 393.0 ± 21.75 | 0.300 ± 0.044 | 16.7 ± 1.08 |

The zeta potential of the developed emulsion was positive due to the presence of cationic polysaccharide (chitosan). The chitosan molecules are localized at the oil-water interface and are intercalated between the nonionic surfactant molecules, either Tween 80 or Poloxamer 188.

The developed emulsions can be considered as a cationic emulsion. Zeta potential was about 22 mV and 16 mV in Tween 80 based emulsions and Poloxamer 188 based emulsion, respectively.

The bio-adhesive property of cationic emulsions of positively charged oil nanodroplets (oily phase) dispersed in water (the continuous phase) can prolong eye drop residence time and improve the bioavailability of drug molecules delivered through these emulsions.

### Example 5

### Stability of Emulsions

Steric (by Poloxamer 188 or Tween 80) and electrostatic (by cationic chitosan) repulsions were responsible for the stabilization of dispersed castor oil droplets in the O/W nano-sized emulsion.

The stability of blank O/W and S/O/W emulsions during the storage period at 4° C. for 1 month was measured in terms of particle size, PDI and zeta potential. Also, the emulsion was visually observed during the storage period.

The emulsions showed no creaming or phase separations.

Results have shown that Tween 80 based emulsions, either blank or loaded with S/O nano-dispersion showed no significant difference in particle size and zeta potential over the study period.

The stability results of Tween 80 and Poloxamer 188 based emulsions over 30 days showed no significant difference in the stability parameters.

### Example 6

### In Vitro Release Studies

In vitro release of AA-2G from S/O/W emulsion was carried out using Float-A-Lyzer® G2 dissolution tubes (molecular weight cut off is 8000-10000 Daltons). About 1 ml of S/O/W emulsion was introduced into the dissolution tubes, which were suspended in about 15 ml of freshly prepared simulated tear fluid (STF; pH 7.4).

The setup was shaken at about 100 RPM in a shaker incubator that was maintained at about 37±0.5° C.

Samples were withdrawn at predetermined time intervals (about 0.5, 1, 2, 4, 6, 8, 24, 32, 48, 54 and 72 hours) and were analyzed using HPLC. The amount of drug released at each time interval was calculated as cumulative percent drug release.

The S/O/W system was able to sustain the drug release for more than 72 hours. Not more than 10.7 and 16.7% of the drug was released during 24 hours from S/O/W-T and S/O/W-P emulsions, respectively. Also, less than 22 and 32% was released during the whole study time from S/O/W-T and S/O/W-P emulsions, respectively.

### Example 7

### In-Vivo Study-Ocular Irritation Test Study (Draize Test)

Ocular irritation test was done to evaluate the tolerance of prepared S/O nano-dispersion as oily eye drops and S/O/W emulsion by using Draize scoring scale.

Two rabbits were used in this experiment for each drug formula in which the formula was introduced into the right eye, whereas the left eye received no treatment and was considered as a control.

Two drops of S/O nano-dispersion as oily eye drops or S/O/W-P emulsion with an approximate volume of 100 μl was placed in the cul-de-sac of the animal right eye.

Discomfort measured as redness and irritation, discharge rate and swelling of cornea and conjunctiva was examined for two days at regular intervals of about 1, 3, 6 and 24 hours.

No discomfort signs were observed for 24 hours. No redness and irritation, no discharge or swelling of cornea and conjunctiva were observed during the study period.

### Example 8

### Corneal Epithelial Healing in Rabbit Eyes

Rabbits were used to evaluate the ability of formulations to accelerate corneal epithelial wound healing in-vivo. Xylazine was used to induce analgesia in the rabbits with intramuscular injections of about 6 mg/kg. Proparacaine 0.5% weight/volume eye drops were also used as supplemental analgesia.

The eyes were kept open during the depredation using a speculum.

Using cotton buds, about 10% of alcohol was applied to the central regions of rabbit corneas for about 20 seconds. Then epithelia within an 8 mm diameter circle in were mechanically scraped using a blunt spatula. After that, eyes were washed several times using normal saline to remove scraped epithelial cells and residual alcohol that can induce corneal irritations.

The rabbits were divided into five treatment groups. Each group contained four animals. Right eyes were selected to receive normal saline while left eyes were assigned to receive the treatment.

The treatment was either about 200 μl (4 drops) of 5% ascorbic acid solution in 0.2 N sodium phosphate buffer pH 7.4, blank oily eye drops, AA as oily eye drops and AA-2G as oily eye drops.

The treatments were administered topically to the wounded eyes, along with topical ofloxacin antibiotic drops (Oflox, Allergan, Inc.), immediately following surgery and 6, 12, 24, 30, 36, 48, 54, 60 and 72-hour post-surgery.

Throughout the healing process, rabbits were closely monitored for evidence of distress or infection, and epithelial wound closure was examined at each dosing time point by applying about 50 μL of 0.25% topical fluorescein eye drops to the injured cornea and imaging the wound under cobalt blue light.

The area of each epithelial defect was measured using “ImageJ”, an image analysis software. Percent healing (“H %”) was calculated as a percentage of the initial wound area. According to the following equation:

$$H \% = \frac{A 0 - A i}{A 0} \times 100 \%$$

Wherein Aᵢ indicates the wound area at a given time point and A₀ indicates the initial wound area.

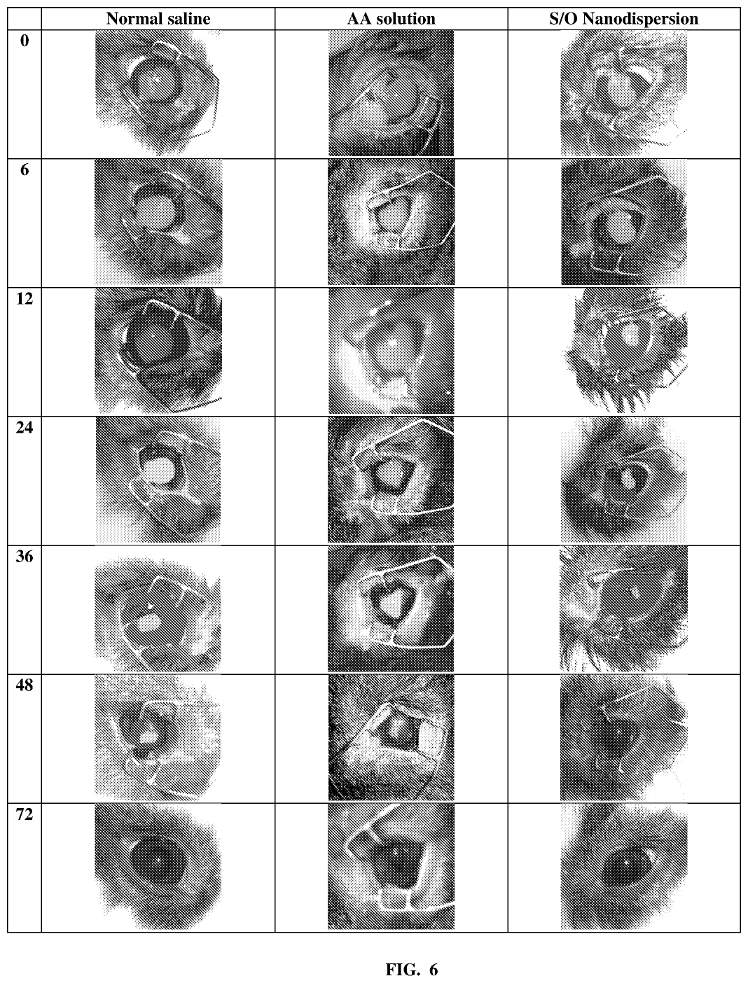

Representative images of the wound area in rabbit corneas following treatment with the normal saline, AA solution and AA loaded S/O nano-dispersion as oily eye drops are shown in FIG. 6.

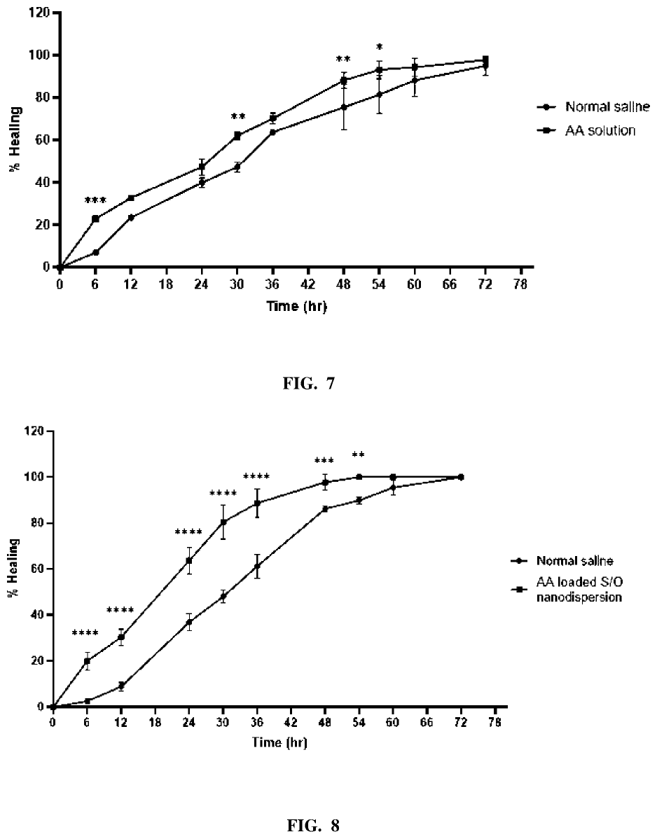

According to FIG. 7, The epithelial wound closure in rabbit corneas was significantly increased in the ascorbic acid solution treated group as compared to the control group (normal saline). Especially at the first 6 hours, % healing was 22.9 vs. 7 (p value 0.0005), the wound-healing rate was enhanced up to 3.28 folds.

The effect of blank oily eye drops as a vehicle of S/O nano-dispersion was also tested. It was shown to have no effect on corneal epithelial healing. The relative healing rate (blank oily eye drops vs normal saline) was almost 1 during the study duration.

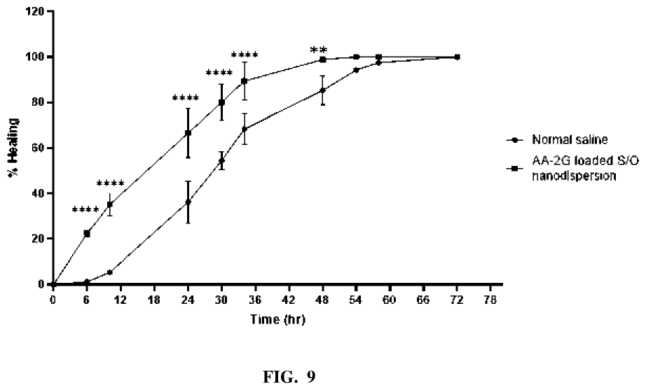

According to FIG. 8, AA loaded S/O nano-dispersion as oily eye drops has a superior effect to accelerate corneal epithelial healing. The wound area in rabbit eyes was significantly smaller in the S/O nano-dispersion treated eyes, than in control eyes at each time point from 6 to 48 hours (p value<0.0001). After 6 hours, % healing was 20.0 vs. 2.2 and the wound-healing rate was enhanced up to 10 folds. In the next 6 hours (after 12 hours), % healing was 30.4 vs. 8.9 and the wound healing rate was enhanced up to 3.8 folds.

Complete wound closure was observed by 48 hours in AA loaded S/O nano-dispersion treated eyes while it needs 72 hours in normal saline treated eyes.

According to FIG. 9, AA-2G loaded S/O nano-dispersion as oily eye drops has a similar effect of AA loaded S/O nano-dispersion.

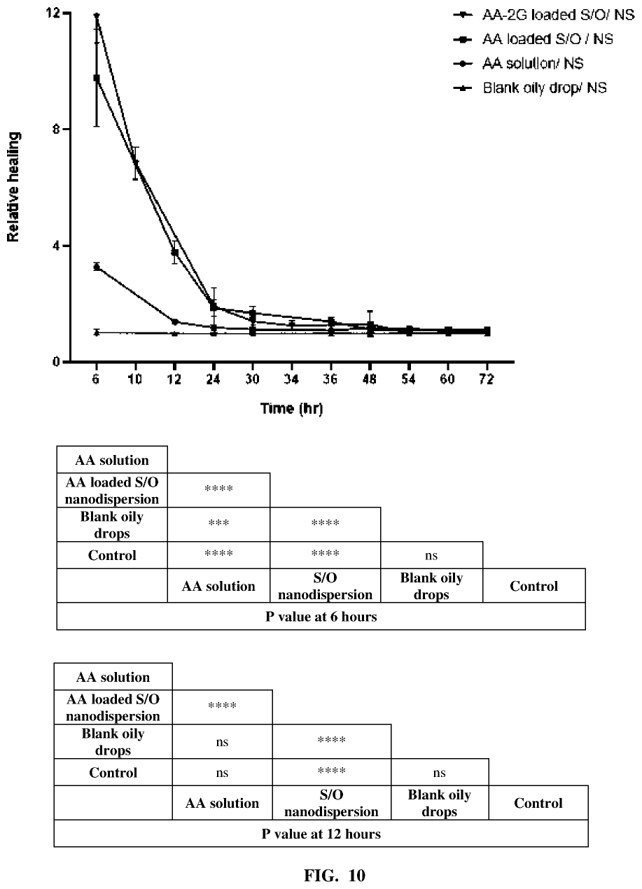

Normalized healing rates and relative healing rates to normal saline, were calculated to compare the effect of the different treatment groups and eliminate the inter-subject variability between each group. As can be seen in FIG. 10, After 6 and 12 hours, S/O nano-dispersion significantly increased the healing rate in comparison with AA solution or normal saline (p value<0.0001). At 6-hour the relative healing rate of AA loaded S/O nano-dispersion, AA solution and normal saline were 9.77 vs. 3.28 vs. 1. At 12-hours, the relative healing rate of AA loaded S/O nano-dispersion, AA solution and normal saline were 3.80 vs. 1.39 vs. 1.

The effect of blank oily drops in the healing process is identical to the effect of normal saline. While the effect of S/O nano-dispersion was significantly different from that of normal saline at each time point from 0-48 hours with the p value less than 0.0001. Also, S/O nano-dispersion significantly different from AA solution at each time point from 6-36 hours, with a p value of <0.0001.

S/O nano-dispersion provides small, hydrophobically modified nanoparticles that can penetrate through the cornea giving improved healing. The enhancement effect in the human could be more prominent considering the fact that's humans are not able to produce AA on their own as rabbits do and they have to get it from diet.

While embodiments of the present disclosure have been described in detail and with reference to specific embodiments thereof, it will be apparent to one skilled in the art that various additions, omissions, and modifications can be made without departing from the spirit and scope thereof.

In describing and claiming the present disclosure, the following terminology will be used.

The singular forms “a,” “an,” and “the” include plural referents unless the context clearly dictates otherwise.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

What is claimed is:

1. A pharmaceutical solid-in-oil nano-dispersion composition comprising ascorbic acid or a stable derivative thereof, a surfactant coating the ascorbic acid or its derivative and an oil vehicle, wherein the ascorbic acid or its derivative are uniformly dispersed in the solid-in-oil nano-dispersion composition.

2. The pharmaceutical solid-in-oil nano-dispersion composition of claim 1, wherein the pharmaceutical composition contains small hydrophobically modified nanoparticles to penetrate through corneal cells.

3. The pharmaceutical solid-in-oil nano-dispersion composition of claim 1, wherein the stable derivative of ascorbic acid is ascorbyl glucoside.

4. The pharmaceutical solid-in-oil nano-dispersion composition of claim 1, wherein the surfactant comprises a hydrophobic surfactant.

5. The pharmaceutical solid-in-oil nano-dispersion composition of claim 4, wherein the hydrophobic surfactant is selected from a group comprising spans, sucrose fatty acid esters, or combinations thereof.

6. The pharmaceutical solid-in-oil nano-dispersion composition of claim 1, wherein the oil vehicle is selected from a group comprising soja oil, castor oil, medium chain triglycerides, long chain truglycerides, Myglyol 812 (triesters of glycerol, capric and caprylic acids), mineral oil and isopropyl myristate.

7. A method of preparing the pharmaceutical solid-in-oil nano-dispersion composition of claim 1, the method comprises the steps of:
Pouring about 10 mL of an aqueous solution containing about 100 mg AA-2G or 52 mg AA and about 20 mL cyclohexane solution containing (0.5, 1, 1.2, or 1.5 g) of a surfactant into a suitable flask;
Mixing the ingredients using a homogenizer at about 24000 round per minute (“rpm”) for about 5 minutes to form a Water in Oil (“W/O”) emulsion;
Freezing the W/O emulsion rapidly using liquid nitrogen;
Lyophilizing the W/O emulsion for about 48 hours to yield a drug-surfactant waxy complex;
Adding a suitable amount of oil vehicle to the resulting complex; and
Dispersing the resulting complex by probe-ultrasonication at about 60 W for about 20 minutes in pulses (pulse on, 15 seconds; pulse off, 15 seconds) to produce S/O nano-dispersions.

8. Use of the pharmaceutical solid-in-oil nano-dispersion composition of claim 1 for healing corneal epithelial wounds.

9. An ointment comprising the pharmaceutical solid-in-oil nano-dispersion composition of claim 1.

10. Oily eye drops comprising the pharmaceutical solid-in-oil nano-dispersion composition of claim 1.

11. A solid-in-oil-in-water emulsion comprising the pharmaceutical solid-in-oil nano-dispersion composition of claim 1.
